(19) United States
(12) Patent Application Publication
(10) Pub. No.: US 2009/0030435 A1
(43) Pub. Date: Jan. 29, 2009

(54) METHOD AND APPARATUS FOR ANCHORING CARDIOVASCULAR IMPLANTS

(75) Inventors: Daniel Rogers BURNETT, San Francisco, CA (US); Shane MANGRUM, Ammon, ID (US)

Correspondence Address: LEVINE BAGADE HAN LLP, 2483 EAST BAYSHORE ROAD, SUITE 100, PALO ALTO, CA, 94303, US

(73) Assignee: TheraNova, LLC, San Francisco, CA (US)

(21) Appl. No.: 12/192,462

(22) Filed: Aug. 15, 2008

Related U.S. Application Data
(63) Continuation-in-part of application No. 11/234,802, filed on Sep. 26, 2005.
(60) Provisional application No. 60/964,822, filed on Aug. 15, 2007.
(60) Provisional application No. 60/613,205, filed on Sep. 27, 2004.

(51) Int. Cl. A61B 17/00; A61B 17/08; A61F 2/04; A61F 2/24

(52) U.S. Cl. 606/153; 606/151; 623/1.36; 623/2.38

(57) ABSTRACT

Methods, devices and systems facilitate retention of a variety of therapeutic devices. Devices generally include an anchoring element, which has been designed to promote fibrotic ingrowth, and an anchored device, which has been designed to firmly engage the complementary region of the anchoring element. The anchoring element may be placed in a minimally invasive procedure temporally separated from the deployment of the anchored device. Once enough time has passed to ensure appropriate fixation of the anchoring element by tissue and cellular ingrowth at the site of placement, the anchored device may then be deployed during which it firmly engages the complementary region of the anchoring element. In this manner, a firm attachment to the implantation site may be made with a minimum of required hardware. Some embodiments are delivered through a delivery tube or catheter and while some embodiments may require laparoscopy or open surgery for one or more of the placement procedures. Some embodiments anchor devices within the cardiovascular tree while others may anchor devices within the gastrointestinal, peritoneal, pleural, pulmonary, urogynecologic, nasopharyngeal or dermatologic regions of the body. An alternative embodiment provides for the placement of the anchoring element and anchored device simultaneously, but allows for their removal separately. This embodiment allows the device, which may be placed only temporarily and be designed to be removed, to experience significant fibrotic ingrowth, but then to be easily detached from the ingrowth-anchored region to allow for simple and quick device removal.

### CROSS-REFERENCE TO RELATED APPLICATIONS

This application claims the benefit of priority to U.S. Prov. Pat. App. 60/964,822 filed Aug. 15, 2007 and is a continuation-in-part of U.S. patent application Ser. No. 11/234,802 filed Sep. 26, 2005 which claims the benefit of priority to U.S. Prov. Pat. App. 60/613,205 filed Sep. 27, 2004, each of which is incorporated herein by reference in its entirety.

### FIELD OF THE INVENTION

The present invention relates to the field of medical devices, in particular therapeutic vascular intervention devices requiring anchoring.

### BACKGROUND OF THE INVENTION

In the last few decades, therapeutic intervention in the cardiovascular arena has seen major advances in the reducing the invasiveness of life-saving procedures. In fact, coronary artery bypass has been surpassed, now, by coronary stenting in most patients with two or fewer lesions. Continuing this trend, several cardiovascular stents and valves have been designed to facilitate minimally invasive placement, frequently through the use of percutaneous catheter technologies.

While successful in several arenas, the minimally invasive placement of cardiovascular devices in areas of high flow or high stress has been relatively unsuccessful. This is due, in large part, to the migration of the devices after they have been placed but before they developed a firm attachment to the wall of the lumen. These issues are currently being seen in several percutaneous aortic valve technologies.

In an effort to combat the migration issue, several devices such as those found in Anduiza U.S. Pat. No. 6,875,231 and Anderson's U.S. Pat. Nos. 6,186,614, 5,840,081 and 5,411,552 are incorporating larger and larger support structures which span larger and larger sections of the lumen, causing issues related to side-branch obstruction and the requirement for anticoagulation. These devices also require a large degree of external pressure in the lumen which they are anchored to be able to withstand the large migration-producing forces they face immediately post-implantation. Furthermore, these issues are also prevalent in percutaneous aortic aneurysm repair and implant anchoring in a variety of locations.

The current state of the art, then, would benefit from a minimally invasive method to firmly anchor cardiovascular and other types of devices in a low-profile, reversible manner using a minimum of hardware. The current invention provides this advance with a two-component procedure in which an anchoring element and an anchored device are placed in separate procedures. This two-part procedure allows for the anchoring element to be placed with enough lead time to allow for cellular ingrowth and firm anchoring prior to placement of the anchored device. Thus, before the device is attached, the physician will be able to visualize exactly where the device will be placed and the device will remain firmly in place once placed. After the anchoring element has been firmly embedded in the vascular wall, the anchored device (stent, valve, etc.) can be inserted and firmly attached to the anchoring element. The insertion of the device can also be accompanied by other interventions, ie native valve debridement, aneurysm decompression, endoscopic intervention, etc. The present invention overcomes the limitations of the prior art by allowing for low-profile insertion into the lumen with a decreased risk of migration due to the presence of extensive tissue-ingrowth prior to exposing the anchored device to the migration-producing forces it will face immediately post-implantation of the anchored device. The present invention also overcomes the limitations of deploying a device with geometrically ellipsoid or circular footprint in a non-geometric implant site which may not form a tight seal with the device (due to normal anatomic variation, calcifications, or other disease processes).

### SUMMARY OF THE INVENTION

As mentioned above, the current invention comprises two components, an anchoring element and the anchored device. When used in combination, the two components provide for the firm, minimally invasive anchoring of hardware within the cardiovascular tree as well as other areas of the body. The anchoring element, itself, has three main functions: 1) to firmly adhere to the lumen, 2) to encourage fibrotic ingrowth, and 3) to provide a firm attachment site for the anchored device. The anchoring element has been designed to adhere to the lumen of the vessel using standard technologies including staples, clips, pins, stents, etc, made from standard materials, nitinol, stainless steel, etc., to provide for a firm attachment. Further examples of anchoring elements are described in detail in U.S. patent application Ser. No. 11/234,802 filed Sep. 26, 2005 (U.S. Pat. App. Pub. 2006/0069400 A1), which is incorporated herein by reference in its entirety. The anchoring element, in the preferred embodiment, is coated with, or fabricated from, materials designed to encourage cellular ingrowth, such as loose weave dacron, polyester, etc, such that with adequate passage of time the element will become firmly embedded in the vessel wall. While this is the preferred embodiment, the present invention doesn't necessarily require this second feature as the benefit of reduction in invasiveness and precision in placement found with this two part device and method make the invention an advance in the field in and of themselves. The cellular ingrowth is anticipated to be beneficial, though, and is considered preferable. The third component of the anchoring element provides for firm attachment of the anchored device. In its preferred embodiment this attachment comprises a reversible mechanical locking mechanism in the medial aspect of the anchoring element. This mechanism, though, could also comprise anchoring provided by magnetism, chemical bonding, interference fit, inflation, etc.

The anchored device can comprise any implanted device requiring firm anchoring. The anchored device comprises the device body, whether it be a mitral valve, aortic valve, aortic aneurysm stent, gastrointestinal stent, etc., which firmly engages the anchoring element. In the case of a lengthy device, such as an aortic aneurysm stent or duodenal sleeve, multiple attachment rings may be placed on the device and multiple anchoring elements may be placed prior to deployment of the device. In this way both the proximal and distal aspects of the device can be firmly anchored once the device is deployed. Optionally, the proximal aspect of the device may be the only portion of the device which attaches to the anchoring element and the distal portion of the device, which requires less mechanical strength, may simply use standard anchoring mechanisms, such as staples, clips, pins, stents, etc.

In its preferred embodiment, then, the anchoring element is placed at the desired site and is then given at least a week (or at least one month or more) to allow for cellular ingrowth, after which the patient then has the device placed. The device then, is held firmly in place using natural cellular and fibrotic reactions thus reducing the requirement for complex and extensive anchoring hardware. The device placement and anchoring element placement, while separate procedures, may be less invasive than any single procedure due to the drastic reduction in required hardware for each procedure. The anchored device addresses a number of the critical issues limiting deployment of existing technologies. Specifically, the anchored device will facilitate precise, secure positioning of the implant without the need for excessive pressures on the wall of the lumen or overly aggressive, but ineffective, anchoring mechanisms at the time of placement of the anchored device. In addition, due to the low-profile, unobtrusive nature of the anchoring element, it may simply be left behind if the device is removed with minimal or no adverse effects on the anchor site. This feature is particularly desirable, in particular, with respect to temporary implants, i.e. gastrointestinal sleeves for obesity, pacing leads for electrical stimulation of bodily tissues, etc., which may be exceeding difficult and laborious to remove from the body once the device is not longer required or becomes defective. One such example includes the use of an anchoring element placed in the vicinity of the pyloric sphincter, either on the sphincter itself or adjacent to the sphincter in the duodenum or the stomach, then allowing time for tissue ingrowth prior to placement of the complementary anchored device, ie gastric or duodenal electrical stimulator, duodenal sleeve, pyloric sphincter restrictor, etc. This mechanism will allow for the placement of gastrointestinal, as well as other, technologies using much less hardware and with a decreased risk of perforation or migration of the anchored device. In addition, the device may be easily removed without major intervention by simply reversing the attachment of the anchored device to the anchoring element which, in this case, involves a firm, but reversible attachment. Due to the uncertain implant times and the fibrotic ingrowth that occurs with these “temporary” devices, the removal procedure may even require an open surgery, at times, and the present invention provides a significant advance in the state-of-the-art by removing this added risk to the patient.

Generally, the time span between the initial implantation of the anchoring element and the subsequent positioning of the device to the anchoring element may range anywhere from a week to a month or more to allow for fibrotic ingrowth such that the implanted device does not shift or migrate once it is placed. For instance, an anchoring ring may be implanted in a patient body and left in place to allow for tissue ingrowth to occur. Several weeks after the implantation of the anchoring ring and the subsequent ingrowth of tissue into and/or around the ring, the anchoring ring may ultimately provide a secure platform for placement of a larger device into the patient body secured by the anchoring ring. Thus, the temporal separation between the initial implantation of the anchoring element and the subsequent implantation and securement of a device to the anchoring element allows for this ingrowth of tissue into and/or around the anchoring element which may provide for secure anchoring within the patient body.

Some additional examples of use which may illustrate particular applications may include the use of inflatable materials to position and hold devices during this ingrowth period. Other examples may also include use of an anchoring ring, use in placement of cardiac or gastric leads, use with temporary and removable devices which may eliminate the need for surgery or lengthy endoscopic procedures, etc. Moreover, such anchoring elements, as well as implantable devices, may be delivered into the patient body through various methods, e.g., percutaneous delivery, transmural delivery, etc.

The device will provide the following advances over the current state of the art: (1) Facilitation of self-seating and avoidance of coronary ostia or other sensitive regions; (2) Elimination of the frequent complication of migration and inadequate sizing with the anchoring of a device with a known diameter or volume to native tissue; (3) Incorporation of radiographic contrast into part one of the anchored device will facilitate localization of the coronary ostia prior to placing the valve and also facilitate exact positioning/orientation of the implant. This feature will also facilitate visualization of real-time fluoroscopic landmarks during placement of the implant; (4) Encouragement of native tissue ingrowth into the anchoring element with the embedding of elements in the anchor material that encourage cellular ingrowth (especially important in the setting of a potentially calcified aorta). This last feature will allow for a greatly decreased footprint of the overall device and use of much less hardware in anchoring the device; (5) Development of a firm, air-tight seal between the device and the bodily tissues due to tissue ingrowth into the anchoring element; (6) Rapid removal of implant, if required, despite the extensive tissue ingrowth even with chronic, long-term implant times.

While the preferred embodiment has been described as an anchoring element to anchor devices within a lumen, the anchoring element may also comprise a ring, a tube, a socket, a port, a catheter, a patch, a clip, a staple, a lead, a button, an inflatable member or any other fastener allowing for consistent contact with bodily tissues in order to allow tissue ingrowth into the anchoring element. The mechanism for firm engagement of the anchoring element to the anchored device may be an inflatable engaging member, interference fit, locking, screw-type or magnetically coupled device, but is not limited to these options as any firm engagement mechanism will suffice.

The interface between the anchored device and the anchoring element may be reversible or irreversible in nature, dependent on the application and the implant requirements. In the case of a temporary implant, for example, the engagement mechanism will be reversible in nature to allow for device removal and, in the event that it is required, replacement of the device as necessary. For permanent implants, the engagement between the anchored device and anchoring element may be irreversible in nature allowing for a more powerful and simpler attachment mechanism.

Also, while the anchored device, in the preferred embodiment, has been described as a cardiovascular device, the anchored device may comprise one or more of several devices including, but not limited to: prosthetic aortic, tricuspid or mitral heart valves, abdominal aortic aneurysm stents, coronary stents, gastrointestinal stents, gastrointestinal devices anchored in the esophagus, stomach or duodenum; gastrointestinal devices anchored within the gastrointestinal lumen, urologic devices anchored within the bladder, peritoneal devices anchored within the peritoneum, pulmonary devices anchored within the pulmonary tree, nasopharyngeal devices anchored within the nasopharynx, orthopedic devices anchored into bone and/or dermatologic devices anchored into the skin or subcutaneous tissues.

Furthermore, in the instance where the anchored device does not experience large forces after placement, the two-stage device can be placed at the same time. In this embodiment useful for devices that are not intended to be permanent or may require replacement, the anchoring ring and anchored device are placed simultaneously in their attached state. As necessary, though, the anchored device may be removed from the anchoring ring and extracted and/or replaced as required. The anchoring ring, though, may remain in place and serve as the point of attachment for an additional device or, in the instance that the device is no longer required, may be left in place or removed.

The competitive advantages of the present invention include: Firm anchoring of device with reduced migration risk, air-tight seal around the implant generated by tissue ingrowth, exact placement of device, reversible nature of device placement and reduced invasiveness for insertion and removal due to reduction in hardware requirements and overall footprint of the implanted device.

### BRIEF DESCRIPTION OF THE DRAWINGS

FIG. 1—Longitudinal section of the anchoring element deployed in the supravalvular position with interference fit and without clips, staples, etc.

FIG. 2—Longitudinal section of the anchoring element deployed in the supravalvular position with clips, staples, etc

FIG. 3—Longitudinal section of the anchoring element deployed in the subvalvular position with clips, staples, etc

FIG. 4—Aerial cross-section view of the anchoring element's entire circumference with clips

FIG. 5A-B—Possible deployment mechanism for anchoring element

FIG. 6A-B—Longitudinal section of the anchoring element deployed in the subvalvular position (FIG. 6A) with clips illustrating fibrotic ingrowth (FIG. 6B).

FIG. 7A-B—Longitudinal section of the anchoring element deployed in the subvalvular position illustrating fibrotic ingrowth (FIG. 7A) and subsequent device deployment and attachment to the anchoring element with debridement of the native valve (FIG. 7B)

FIG. 8A-B—Longitudinal section of the anchoring element deployed in the subvalvular position illustrating fibrotic ingrowth (FIG. 8A) and subsequent device deployment and attachment to the anchoring element without debridement of the native valve (FIG. 8B)

FIG. 9—Longitudinal section of the anchoring element deployed in the subvalvular position illustrating fibrotic ingrowth (FIG. 8A) and subsequent deployment of porcine valve to the anchoring element with debridement of the native valve (FIG. 8B)

FIG. 10A-C—Longitudinal section of the anchoring element deployed with clips in the abdominal aorta (FIG. 10A) illustrating fibrotic ingrowth (FIG. 10B) and subsequent deployment of abdominal aortic aneurysm stent to anchoring element (FIG. 10C)

FIG. 11—Cross-section of the gastrointestinal tract anchoring embodiment illustrating anchoring of a lower esophageal ring and a pyloric ring to which a intestinal sleeve is attached

FIG. 12—Cross-section of the gastrointestinal tract anchoring embodiment wherein an electrical stimulator for the treatment of obesity is shown attached to a pyloric anchoring element

FIG. 13A-C—Deployment of inflatable anchoring element

FIG. 14A-C—Deployment of stapled or clipped anchoring ring

FIG. 15A-C—Deployment of electrical lead with anchoring element, i.e. a gastric or cardiac lead

FIG. 16A-C—Placement and removal of a temporary anchored device, i.e. a gastrointestinal drug pump

FIG. 17A-C—Side view of percutaneous anchored device placement at site of anchoring element

FIG. 18A-C—Top-view of deployment of the anchored device onto the anchored ring

FIG. 19—Transmural placement of the anchoring element

### DETAILED DESCRIPTION OF THE INVENTION

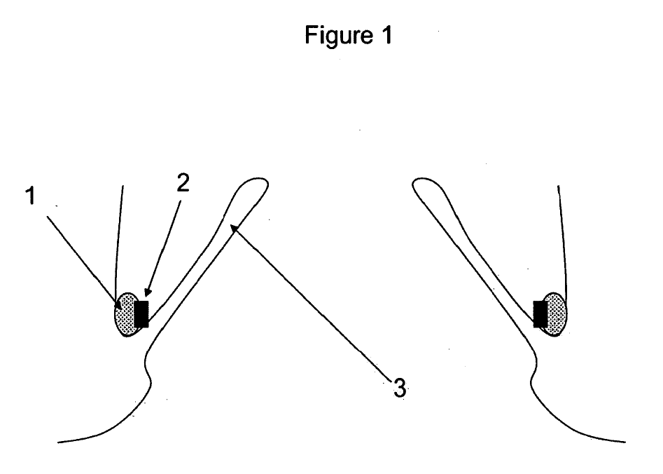

FIG. 1—This illustration represents the deployed anchoring element placed in the aortic valve region. As can be seen from the illustration, the anchoring element comprises a material to promote fibrotic ingrowth 1 and the attachment ring 2 designed to engage the aortic valve device. In this case, the device is deployed above the native aortic valve 3.

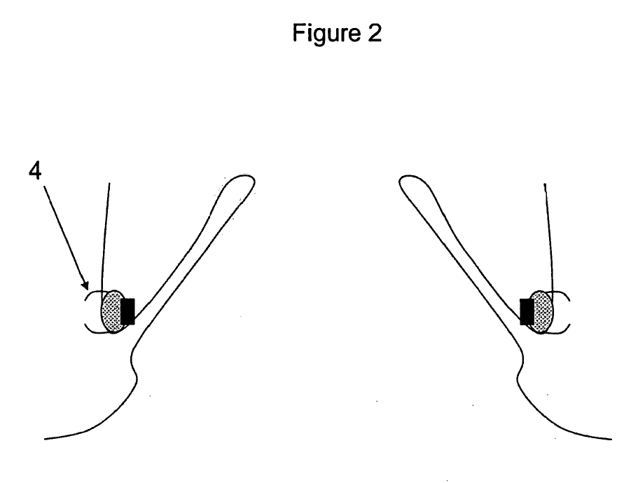

FIG. 2—This illustration represents the deployed anchoring element again placed in the aortic valve region but this time with clips to secure the anchoring element to the aorta. As can be seen from the illustration, the anchoring element comprises a material to promote fibrotic ingrowth 1, the attachment ring 2 designed to engage the aortic valve device and clips, staples, etc 4 for the attachment of the anchoring element to the aortic tissues. In this case, the device is deployed above the native aortic valve.

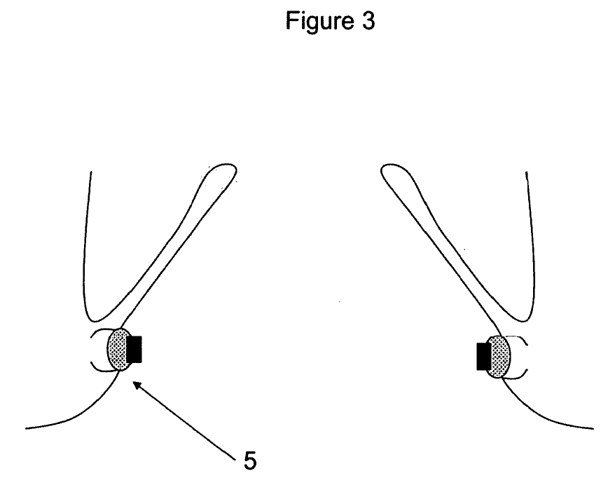

FIG. 3—This illustration represents the deployed anchoring element again placed in the aortic valve region but this time with clips to secure the anchoring element. As can be seen from the illustration, the anchoring element comprises a material to promote fibrotic ingrowth 1, the attachment ring 2 designed to engage the aortic valve device and clips, staples, etc 4 for the attachment of the anchoring element to the aortic tissues. In this case, the device is deployed below the native aortic valve.

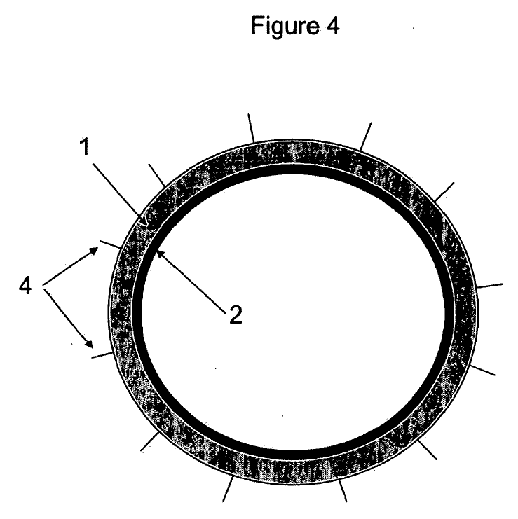

FIG. 4—Is an aerial cross-section view of the anchoring element's entire circumference with clips. In this view the entire circumference of the fibrotic ingrowth anchoring element 1 and the device attachment ring 2 can be visualized as can the clips 4 to secure the device to native tissues. In this case the anchoring element is attached with clips, but it could be attached with a expanding stent, staples, sutures, glues or other attachment modalities.

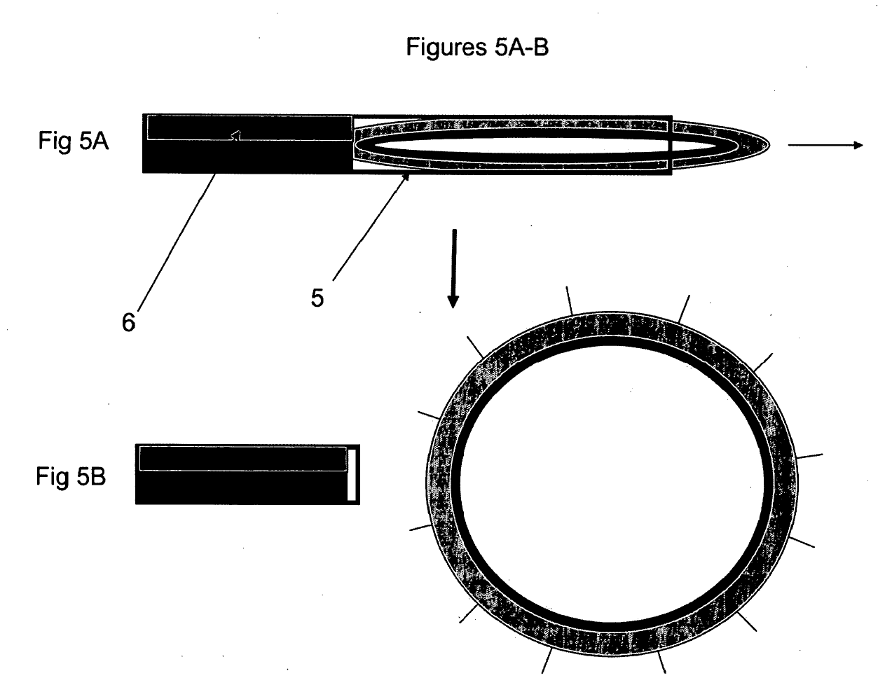

FIG. 5A-B—Is a view of a possible deployment mechanism for the anchoring element. In this case the anchoring element is placed inside of an insertion tube or catheter 5 and placed in the region where deployment is desired. A plunger 6 is then used to expel the device into its proper position. This is but one of many possible deployment mechanisms with the optimal embodiment being a deployment mechanism that allows for reversible circumferential deployment in a consistent manner.

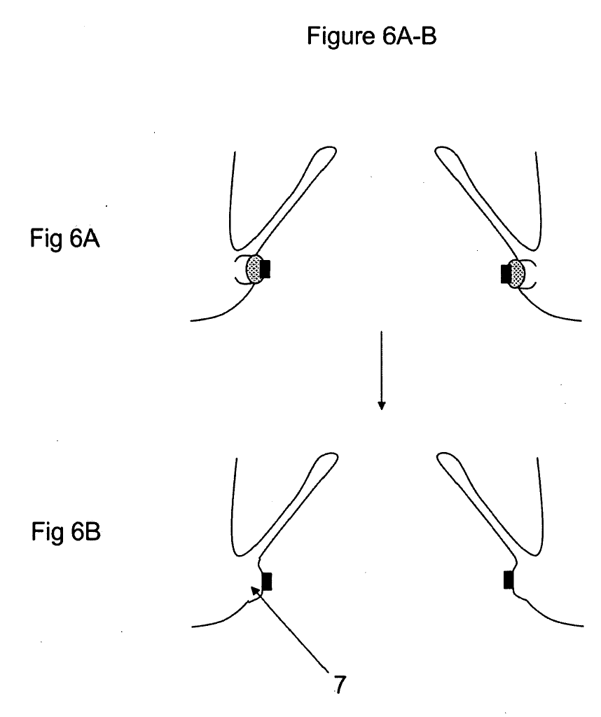

FIG. 6A-B—Longitudinal section of the anchoring element deployed in the subvalvular position (FIG. 6A) with clips illustrating fibrotic ingrowth (FIG. 6B). In this case, the subvalvular clips hold the anchoring element in place until the cellular and fibrotic ingrowth can provide for firm permanent attachment 7.

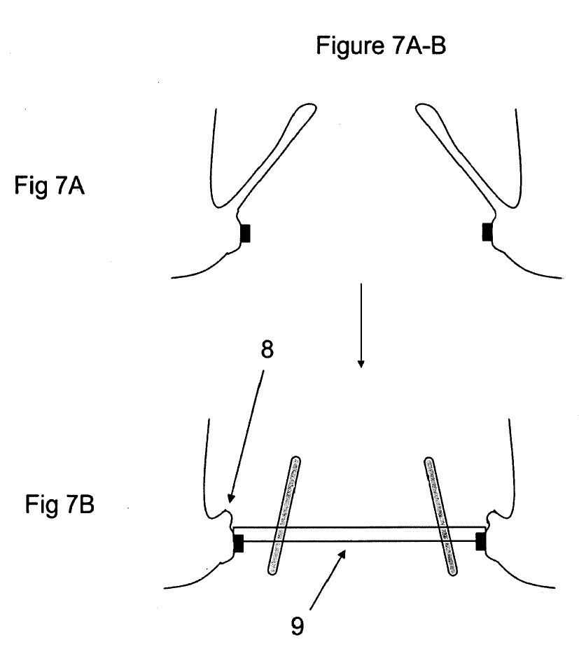

FIG. 7A-B—In this longitudinal section of the anchoring element deployed in the subvalvular position fibrotic ingrowth (FIG. 7A) and subsequent device deployment to the anchoring element with debridement of the native valve (FIG. 7B) are illustrated. Once the anchoring element has been firmly anchored by fibrotic ingrowth, the new aortic valve 9 may be placed with or without removal of the existing native valve 8.

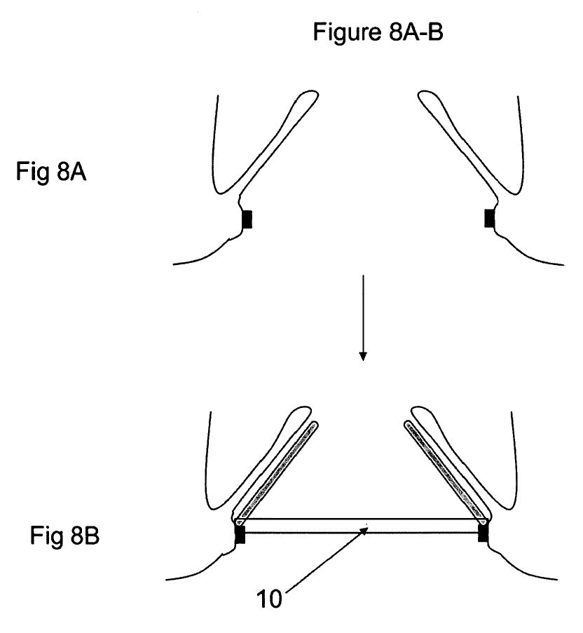

FIG. 8A-B—Longitudinal section of the anchoring element deployed in the subvalvular position illustrating fibrotic ingrowth (FIG. 8A) and subsequent device deployment to anchoring element without debridement of the native valve (FIG. 8B). Here the aortic valve device 10 is placed without removal of the native valve.

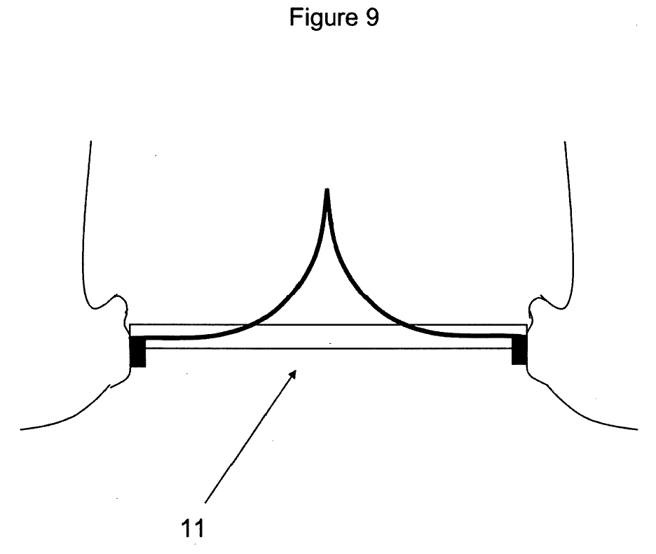

FIG. 9—Longitudinal section of the anchoring element deployed in the subvalvular position illustrating fibrotic ingrowth (FIG. 8A) and subsequent deployment of porcine valve 11 to anchoring element with debridement of the native valve (FIG. 8B)

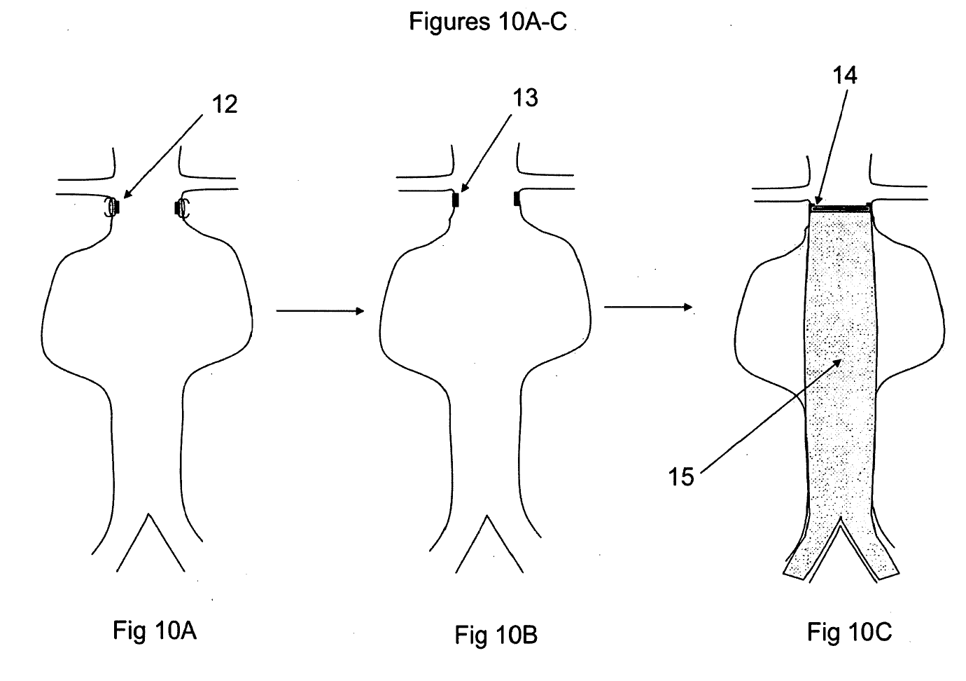

FIG. 10A-C—Longitudinal section of the anchoring element deployed with clips in the abdominal aorta 12 (FIG. 10A) illustrating fibrotic ingrowth 13 (FIG. 10B) and subsequent deployment of abdominal aortic aneurysm stent 15 to the anchoring element 14 once said ingrowth has occurred (FIG. 10C). In this case the anchoring element has been deployed just below the renal arteries and just above the neck of the aneurysm. As can be seen in this case, this invention allows for much more precise placement of abdominal aortic stents in much tighter regions than would otherwise be possible with existing longer stent-based devices.

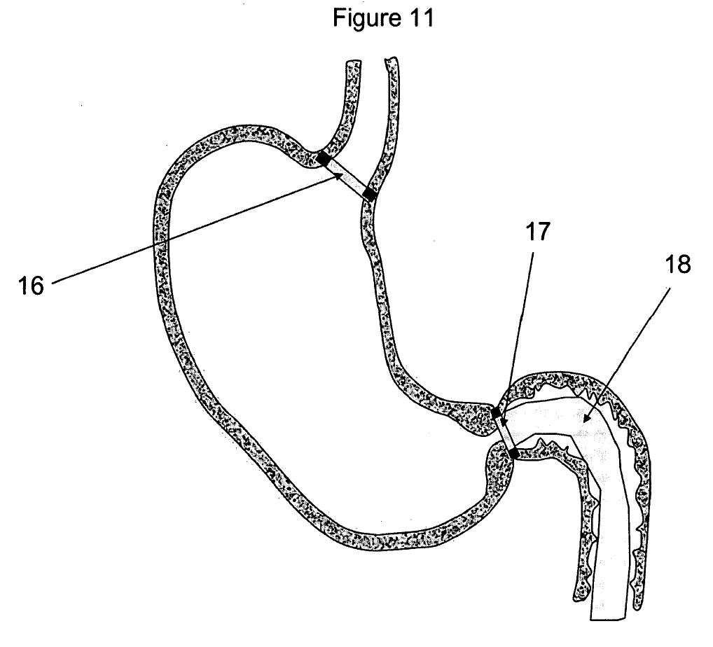

FIG. 11—Cross-section of the gastrointestinal tract anchoring embodiment illustrating anchoring of a lower esophageal ring 16 and a pyloric ring 17 to which an intestinal sleeve is attached 18. These are but two of the possible embodiments of the gastrointestinal devices that can be anchored using this technology with other possibilities including, but not limited to: an intestinal sleeve, an electrical stimulator designed to alter transit or treat obesity, for an artificial rectum, a gastric pouch for the treatment of obesity and a flow restrictor for gastrointestinal transit.

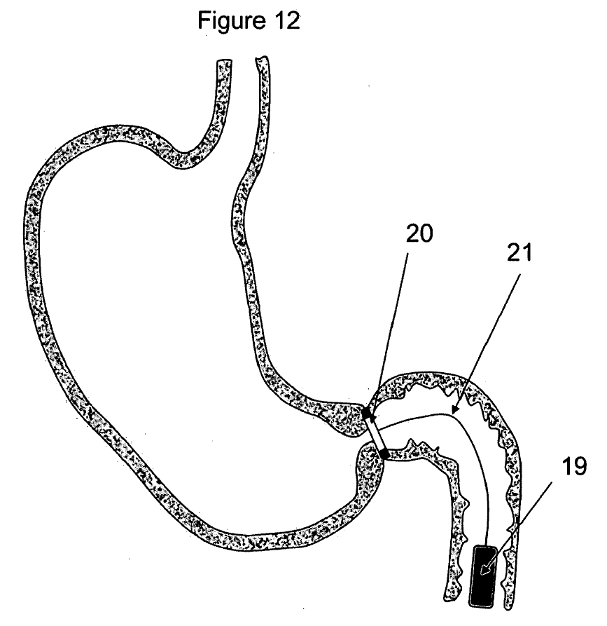

FIG. 12—Cross-section of the gastrointestinal tract anchoring embodiment wherein an electrical stimulator for the treatment of obesity 19 is shown anchored to the pyloric anchoring element 20, in this case via an optional conducting tether 21. The anchoring element may be in the pyloric, cardiac or fundic regions of the stomach or may be attached to the esophagus or intestine. The tether may not be used in the instance where the stimulator is low-enough profile that it will not overly impede flow through the gastrointestinal tract and cause a bowel obstruction. The anchoring element may comprise the ring illustrated, but may also comprise any configuration of ingrowth encouraging material (including stapling, suturing, etc, at a single, non-circumferential site) deployed in any region of the stomach, esophagus or intestine. In the preferred embodiment, the anchoring element is deployed in a non-mucosal region of the stomach, esophagus and/or intestine in order to avoid erosion, ulceration and/or rupture.

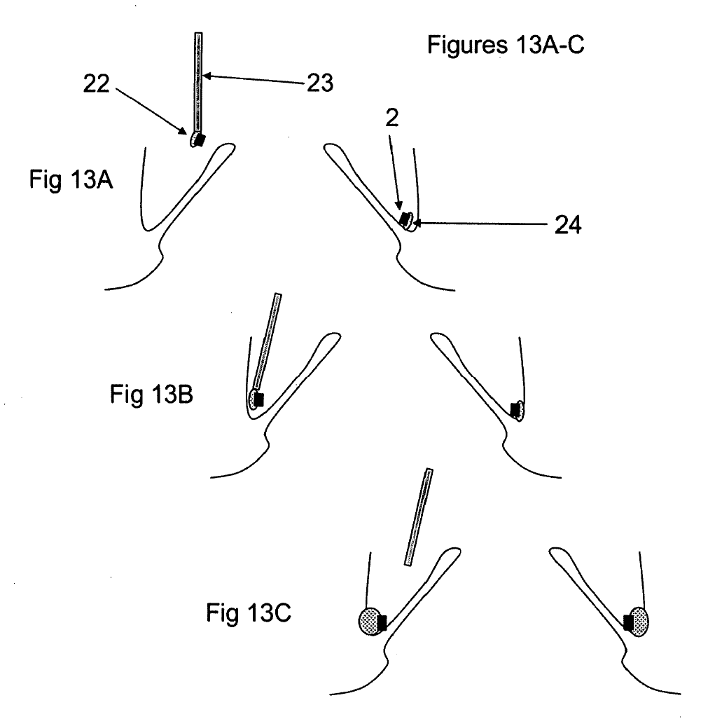

FIG. 13A-C—Deployment of inflatable anchoring element. In this embodiment, the anchoring ring 22 is illustrated being deployed from a catheter 23 (FIG. 13A), positioned in its desired location, in this case the aortic root, (FIG. 13B) and then inflated or expanded to provide snug fit and close approximation to the bodily tissues after which the positioning/inflation catheter 23 is detached (FIG. 13C). The inflatable portion of the anchoring ring 24 may be filled with saline, air, oil, a polymerizing material, a shape-memory polymer or any other material that will allow it to hold its shape. The inflatable member may hold its shape for a minimum of 1 week after placement to ensure adequate ingrowth prior to loss of inflation support. The same mechanism may be used to provide a firm engagement of the anchored device by the anchoring ring wherein the anchored device utilizes an inflatable member which, when correctly positioned and filled, will firmly attach the anchored device to the anchoring ring. In this illustration, the attachment ring 2, may comprise any embodiment, though, as long as it provides from attachment of the anchoring ring to the anchored device.

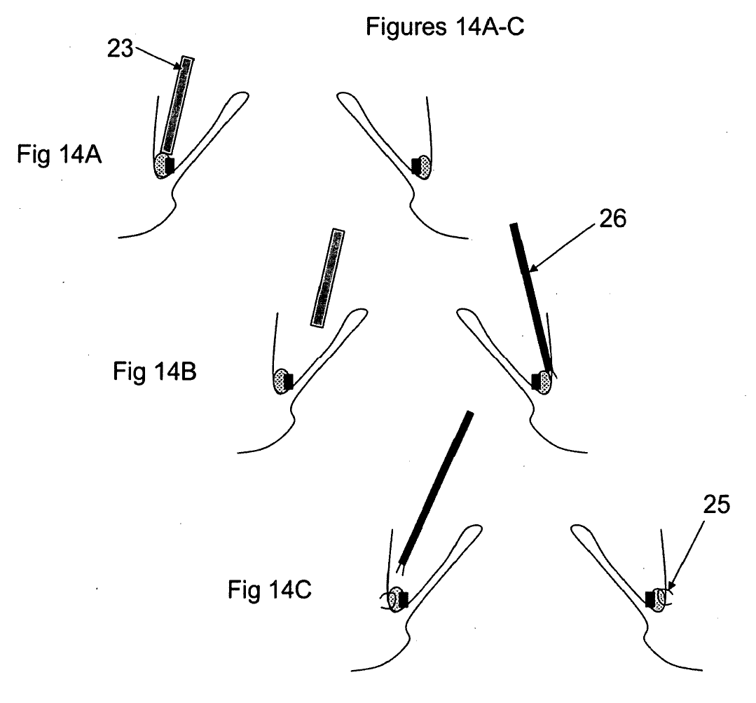

FIG. 14A-C—Deployment of stapled or clipped anchoring ring. In this illustration, the anchoring ring is shown being deployed via catheter 23 at the site of placement using and then stapled or clipped in place. In this embodiment, the clips, pins, barbs or staples 25 are not a part of the anchoring ring, but are deployed using a percutaneous catheter 26 by the physician once the device is in position. The anchored device then engages the attachment ring 2 as previously described once sufficient tissue ingrowth has occurred to firmly anchor the device in place and to provide a circumferential seal.

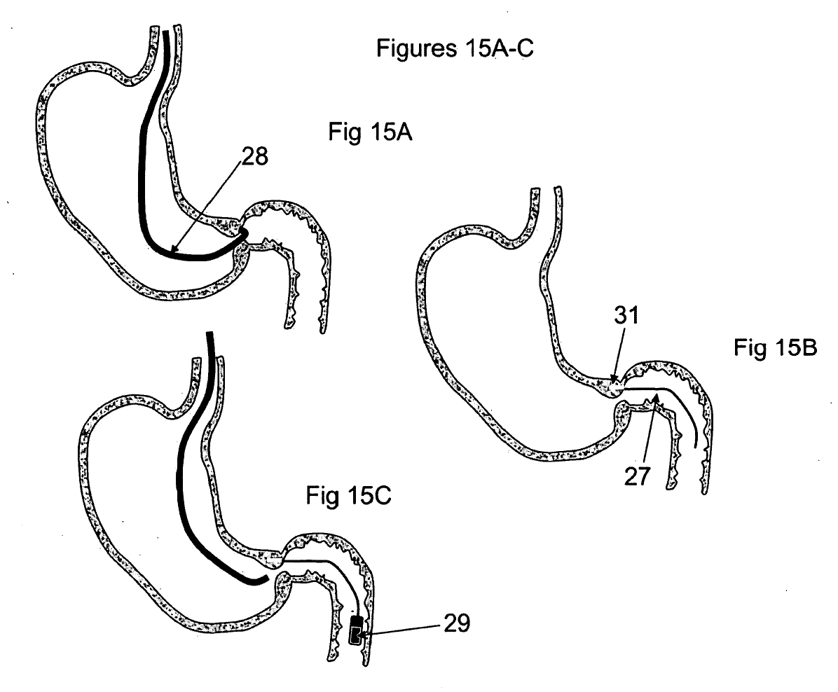

FIG. 15A-C—Deployment of electrical lead with anchoring element, i.e. a gastric or cardiac lead. In FIG. 15A, a gastric lead 27 is shown being placed in the distal pylorus via an endosccope 28 in order to provide firm attachment and encourage ingrowth to the anchoring element 31 prior to intraluminal placement of the anchored device 29. The lead may be placed anywhere in the gastrointestinal tract, though, and is not limited to only the distal pylorus. Once the lead has been placed and sufficient time has elapsed to allow for tissue ingrowth into the anchoring element 31, the anchored device 29 may be deployed attached to the gastric lead 27. In this embodiment, an anchoring element may be used, not an anchoring ring, and this anchoring element may be used anywhere in the body that firm, chronic lead placement is desired. In either case where a gastric or cardiac lead is placed within the patient body, a low-profile anchoring element may be implanted which is able to withstand the environmental forces subjected upon the anchor. Given enough time through the temporal separation to allow for the tissue ingrowth into and/or around the anchoring element, the device may be subsequently attached or otherwise coupled to the anchoring element providing a stabilized anchoring platform.

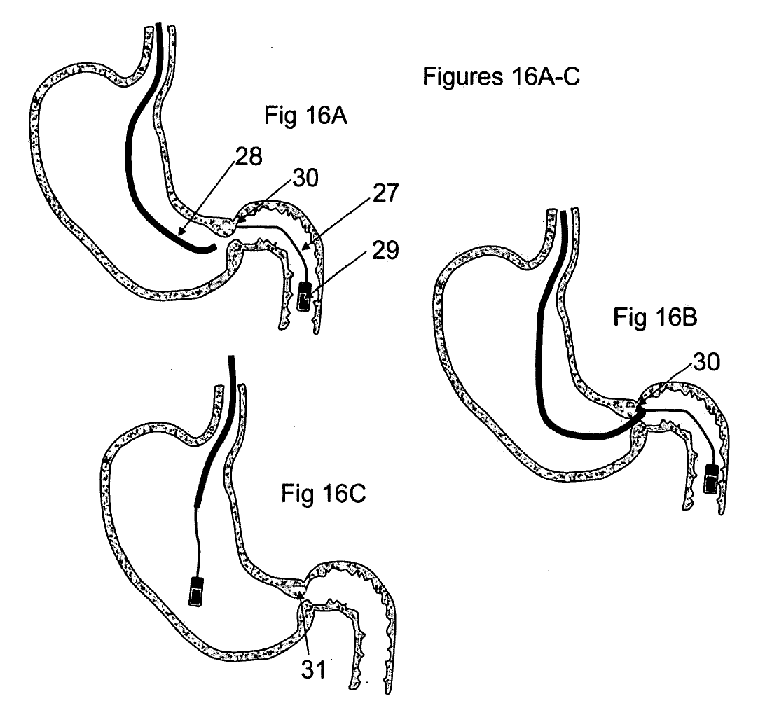

FIGS. 16A-C—Removal of a temporary device, e.g. a gastrointestinal drug pump. The intestinal device 29 attached to the distal pylorus from FIG. 15 is illustrated here in which a simple detachable element 30 on the lead 27 allows for easy removal of the anchored device. In FIG. 16A the anchored device 29 and anchoring element 27 of FIG. 15 is shown being placed, except in this embodiment the anchoring element 27 has a detachment element 30 near its insertion site at the pylorus. The anchored device 29 may be placed along with the anchoring element 27 and the two-part system may be used to facilitate ease of removal in addition to firm attachment. In FIG. 16 B, the anchored device 29 is shown being removed after detaching the anchored device 29 from the anchoring element 31, at the detachable element 30 via endoscopy which otherwise would require a much more elaborate procedure or surgery in order to fully remove due to the extensive and intentional ingrowth. The portion of the anchoring element that has experienced significant fibrotic ingrowth 31 may then be left in situ, as illustrated in FIG. 16C, where it will remain as a benign area of fibrotic ingrowth and may even be reused for replacement of the anchored device 29 in the future. The benefit of this feature is that the removal of the device after firm anchoring by fibrotic ingrowth may be quite difficult and/or impossible via endoscopic techniques. This feature allows the anchored device 29 to be removed quickly and easily while leaving the firmly anchored element 31 behind without the requirement for surgical removal or lengthy endoscopic or laparoscopic procedures. This temporally spaced or simultaneous placement of the detachable anchoring element and anchored device with intended detachment of the anchored device from the anchoring element for removal may be employed with any of the embodiments listed above and, in particular, with the embodiments illustrated in FIGS. 11, 12 and 15 in which the anchored device may require temporary, but firm, placement. The simultaneous placement of the anchored device 29 and the anchoring element 31 with a detachment point 30 for ease of removal is particularly useful in low-stress applications where the fibrotic ingrowth is useful for chronic placement, but not necessary for acute placement. Suture or clips may then be used to temporarily anchor the device prior to tissue ingrowth providing firm anchoring. These examples are provided as illustrations and are not intended to be limiting. Generally, even in procedures where devices may be easily placed, e.g., within the gastrointestinal tract, heart, vasculature, etc., the devices are frequently difficult to remove from the patient body. By allowing for the disengagement from the anchoring element which is firmly secured within the body, the device may be readily detached from the anchoring element and removed from the body while leaving the anchoring element behind in an unobtrusive manner.

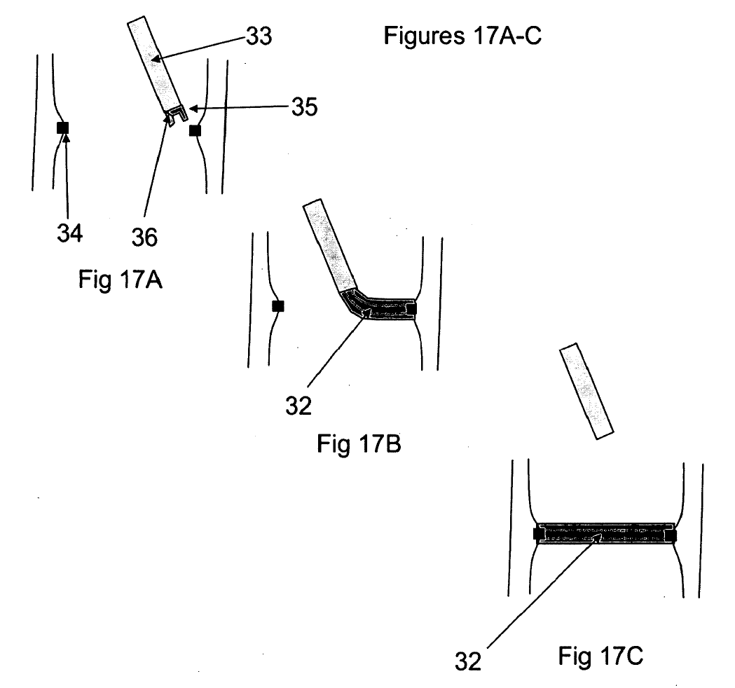

FIGS. 17A-C—Side view of percutaneous anchored device placement at site of anchoring element. Generally, anchoring elements in this variation may be delivered and/or deployed within a patient body by minimally invasive access procedures. Such examples may include percutaneous or laparoscopic access within the patient body by catheter or laparoscopic instruments where access to targeted regions may be accomplished without the need for open surgical access. In this illustration, the anchored device 32 is shown being deployed from a percutaneous catheter 33 at the site of the anchoring element 34 after sufficient fibrotic ingrowth has occurred (FIG. 17A). In FIG. 17B, a side-view is shown of the anchored device 32 being deployed with a reversible locking grasp tip 35 which may include a locking hinge 36 to allow for firm locking to the anchoring ring 34 once fully deployed, as in FIG. 17C. One or more of these locking graspers 35 or locking levers may be deployed at intervals throughout the anchored device 32 or may be incorporated into the anchoring element 34. Alternatively, the device may be appropriately sized to allow for an interference fit without the requirement for one or more locking graspers 35. Alternatively, as well, the grasp tip 35, may include a magnetic element in addition to, or instead of, the hinged grasper 36 with complementary, oppositely charged magnetic sites on the anchoring element 34. These are but a few of the embodiments and should not restrict the final design from incorporating other embodiments to provide for engagement of the anchored device 32 with the anchoring ring 32. The illustrated design is that of the aortic valve repair or replacement, but the attachment elements described may be utilized in any of the many embodiments for the implant anchoring technology described.

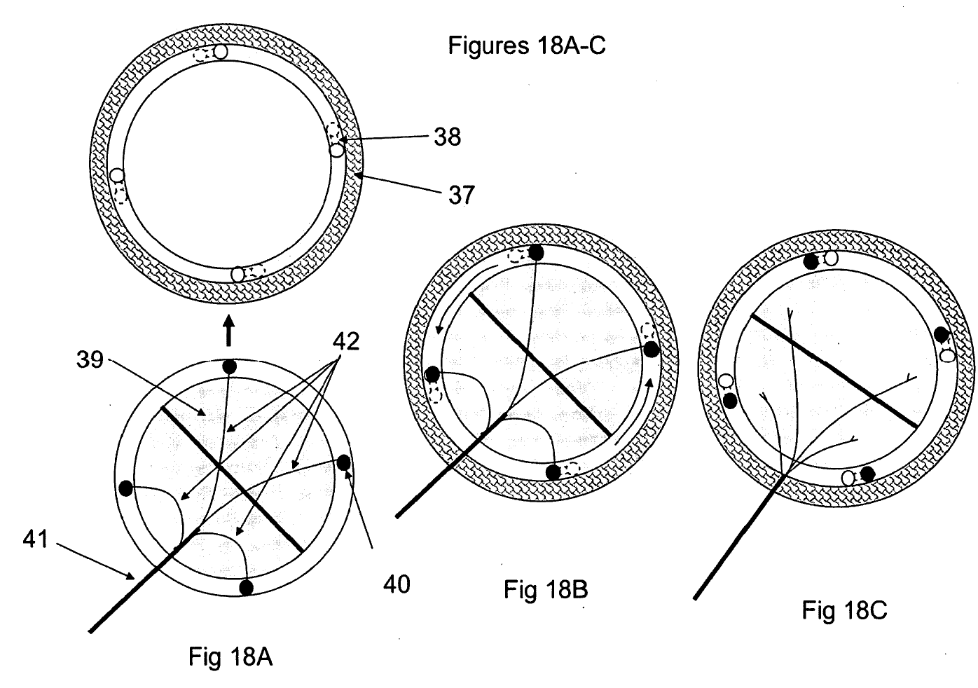

FIG. 18A-C—Top-view of deployment of the anchored device onto the anchored ring. In these figures, an anchored device is seen engaging an anchoring ring via percutaneous manipulation. The anchoring ring can be seen with its ingrowth encouraging fabric incorporated into a standard expanding slotted tube stent 37 and with four attachment points 38 which allow the anchored device 39, which is in this case a bi-leaflet cardiac valve, to be firmly secured. In this case, the firm attachment points 38 are shown as ball and socket sliding and locking mechanisms, but they may incorporate any of the above-mentioned attachment mechanisms, as well as ultrasonic and EMF welding. The complementary attachment points 40 (the ball for the ball and sliding socket) are shown connected to an insertion/deployment catheter 41 in this case via individual catheters 42 which may provide torque or provide other manipulation for placement and locking as in FIG. 18B once the device is in position. Once placed, the individual attachment catheters 42 may be released leaving the device snugly in place with a lower-profile insertion footprint than ever previously possible without surgery.

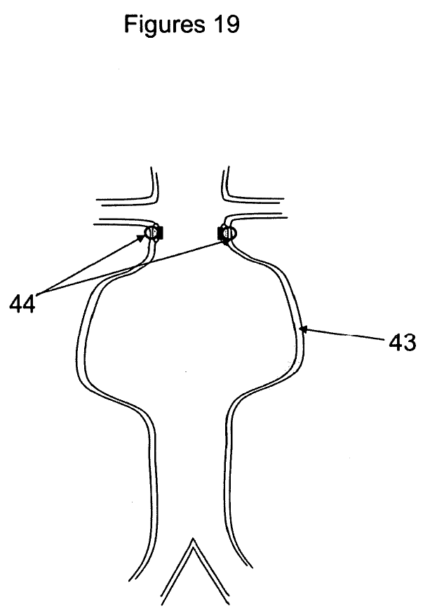

FIG. 19—Transmural placement of the anchoring element. In this illustration, an embodiment employing transmural anchoring (with clips, pins, staples, sutures, RF, microwave or ultrasonic energy) is illustrated. The anchoring elements 44 designed to hold the anchoring ring in place prior to sufficient tissue ingrowth to securely attach the device span the wall 43 of the insertion lumen in order to both anchor the device more firmly (acutely and chronically) and to prevent progression of disease within the wall, in this case in the form of an expanding aneurysm. By placing the anchoring elements transmurally, relatively more robust fibrotic tissue ingrowth may be encouraged resulting in a more secure anchoring of the device. A sufficient fibrotic ring may prevent tracking of the aneurysm along the vessel which in this case would lead to involvement of the renal arteries with usually dire consequences.

What is claimed is:

1. A multi-component implant anchoring device comprising:
an anchoring element; and
an anchored device, wherein
said anchoring element and anchored device are inserted separately and form firm attachments to each other upon deployment within the body and wherein,
said anchoring element incorporates a material or fabric to encourage cellular ingrowth and wherein,
said anchoring element is inserted a period of time prior to the insertion of the anchored device to allow for fibrotic or cellular ingrowth to firmly secure the element in place and wherein,
said anchored device contains at least one complementary attachment site for the anchoring element allowing for firm attachment upon implantation and wherein,
said anchored device comprises one or more of the following devices: prosthetic aortic, tricuspid or mitral heart valves, abdominal aortic aneurysm stents, coronary stents, gastrointestinal stents, electrical gastric stimulators, electrical intestinal stimulators, gastrointestinal devices anchored in an esophagus, stomach, intestine or rectum, urogynecologic devices anchored within a bladder, uterus, fallopian tubes or vagina, peritoneal devices anchored within a peritoneum, pulmonary devices anchored within a pulmonary tree, nasopharyngeal devices anchored within a nasopharynx, orthopedic devices anchored into bone and/or dermatologic devices anchored into skin.

2. A multi-component implant anchoring device comprising:
an anchoring element and
an anchored device, wherein
said anchoring element and anchored device are inserted separately and form firm attachments to each other during deployment inside the body.

3. The device of claim 2 in which said anchoring element contains support elements to provide for firm attachment to the surrounding tissues at the site of implantation wherein said support elements comprise pins, staples, clips, stents, sutures, inflatable members, barbs, or shape-memory materials.

4. The device of claim 2 in which said anchoring element incorporate a material or fabric to encourage cellular ingrowth.

5. The device of claim 4 in which said fabric comprises a Dacron, polyester, Gore-Tex, PTFE or other ingrowth encouraging material.

6. The device of claim 2 in which said anchored device comprises at least one complementary attachment site for the anchoring element.

7. The device of claim 6 in which said anchoring ring provides for firm attachment of said anchored device through the use of an inflatable engaging member, shape-memory expansion, screwing, mechanical locking, magnetic attraction, chemical bonding, or interference fitting.

8. The device of claim 6 in which said anchoring ring provides for firm attachment to said anchored device through the use of pins, staples, clips, stents, or sutures.

9. The device of claim 2 in which said anchoring element and said anchored device can each be collapsed to a size sufficient to allow for minimally invasive, catheter-based delivery.

10. The device of claim 2 in which radiographic contrast is incorporated into said anchored device to facilitate localization of anatomic landmarks (such as the coronary ostia) prior to placing said anchored device and also facilitate exact positioning/orientation of the implant.

11. The device of claim 2 in which said anchored device encourages native tissue ingrowth into the anchor with the embedding of elements in the anchor material that would encourage cellular ingrowth.

12. The device of claim 2 in which the attachment of said anchoring device to said anchoring element is accompanied by another intervention comprising native valve debridement for percutaneous aortic valve repair.

13. The device of claim 2 wherein said anchored device comprise one or more of the following devices: prosthetic aortic, tricuspid or mitral heart valves, abdominal aortic aneurysm stents, coronary stents, gastrointestinal stents, gastrointestinal devices anchored in the esophagus, stomach or duodenum, gastrointestinal devices anchored within the gastrointestinal lumen, urogynecologic devices anchored within the bladder, uterus, fallopian tubes or vagina, peritoneal devices anchored within the peritoneum, pulmonary devices anchored within the pulmonary tree, nasopharyngeal devices anchored within a nasopharynx, orthopedic devices anchored into bone and/or dermatologic devices anchored into skin.

14. A method of anchoring an implanted device comprising:
placing an anchoring element; and
deploying the anchored device wherein
Said anchoring element firmly engages said anchored device.

15. The method of claim 14 in which at least a portion of said anchoring element encourages native tissue ingrowth and/or cellular ingrowth.

16. The method of claim 14 in which said anchoring element comprise a ring, a tube, a socket, a port, a catheter, a patch or any other fastener allowing for firm engagement of the complementary region of the anchored device.

17. The method of claim 14 in which said anchoring element contain support elements comprising staples, clips, stents, or sutures to provide for firm attachment to the surrounding tissues at the site of implantation prior to said native tissue ingrowth and/or cellular ingrowth.

18. The method of claim 15 in which at least three days, at least a week, at least a month and/or at least three months is allowed to pass between the placement of said anchoring element and the deployment of said anchored device.

19. The method of claim 14 in which said anchoring ring provides for firm attachment to said anchored device through the use of screwing, mechanical locking, magnetic attraction, chemical bonding, interference fitting, suturing, stapling, or clipping.

20. The method of claim 14 wherein said anchored device may comprise one or more of several devices including: prosthetic aortic, tricuspid or mitral heart valves, abdominal aortic aneurysm stents, coronary stents, gastrointestinal stents, electrical gastric stimulators, electrical intestinal stimulators, gastrointestinal devices anchored in an esophagus, stomach or duodenum, gastrointestinal devices anchored within a gastrointestinal lumen, urogynecologic devices anchored within a bladder, uterus, fallopian tubes or vagina, peritoneal devices anchored within a peritoneum, pulmonary devices anchored within a pulmonary tree, nasopharyngeal devices anchored within a nasopharynx, orthopedic devices anchored into bone and/or dermatologic devices anchored into skin.

21. A multi-component implant anchoring device comprising:
an anchoring element; and
an anchored device, wherein
said anchoring element and anchored device are inserted separately and form firm but reversible attachments to each other during deployment inside the body.

22. The device of claim 21 in which said anchoring element contains support elements to provide for firm attachment to the surrounding tissues at the site of implantation wherein said support elements comprise pins, staples, clips, stents, sutures, inflatable members, barbs, or shape-memory materials.

23. The device of claim 21 in which said anchoring element incorporates a material or fabric to encourage cellular ingrowth.

24. The device of claim 23 in which said fabric comprises a Dacron, polyester, Gore-Tex, PTFE or other ingrowth encouraging material.

25. The device of claim 21 in which said anchored device comprises at least one complementary attachment site for the anchoring element.

26. The device of claim 25 in which said anchoring ring provides for firm attachment of said anchored device through the use of an inflatable engaging member, shape-memory expansion, screwing, mechanical locking, magnetic attraction, chemical bonding, or interference fitting.

27. The device of claim 25 in which said anchoring ring provides for firm attachment to said anchored device through the use of pins, staples, clips, stents, or sutures.

28. The device of claim 21 in which said anchoring element and said anchored device can each be collapsed to a size sufficient to allow for minimally invasive, catheter-based delivery.

29. The device of claim 21 in which radiographic contrast is incorporated into said anchored device to facilitate localization of anatomic landmarks prior to placing said anchored device and also facilitate exact positioning/orientation of the implant.

30. The device of claim 21 in which said anchored device encourages native tissue ingrowth into the anchor with the embedding of elements in the anchor material that encourages cellular ingrowth.

31. The device of claim 21 in which the attachment of said anchoring device to said anchoring element is accompanied by another intervention, comprising native valve debridement for percutaneous aortic valve repair.

32. The device of claim 21 wherein said anchored device comprise one or more of the following devices: prosthetic aortic, tricuspid or mitral heart valves, abdominal aortic aneurysm stents, coronary stents, gastrointestinal stents, gastrointestinal devices anchored in an esophagus, stomach or duodenum, gastrointestinal devices anchored within a gastrointestinal lumen, urogynecologic devices anchored within a bladder, uterus, fallopian tubes or vagina, peritoneal devices anchored within a peritoneum, pulmonary devices anchored within a pulmonary tree, nasopharyngeal devices anchored within a nasopharynx, orthopedic devices anchored into bone and/or dermatologic devices anchored into skin.

33. The device of claim 21 in which the attachment of said anchoring device to said anchoring element is reversible allowing for detachment of the anchored device from the anchoring ring and removal of the anchored device from the body while leaving the firmly embedded anchoring ring in situ.

34. A multi-component implant anchoring device comprising:
an anchoring element; and
an anchored device, wherein
said anchoring element and anchored device are inserted simultaneously, but removed separately, and form firm but reversible attachments to each other during deployment inside the body.

35. The device of claim 34 in which said anchoring element contains support elements to provide for firm attachment to the surrounding tissues at the site of implantation wherein said support elements comprise pins, staples, clips, stents, sutures, inflatable members, barbs, or shape-memory materials.

36. The device of claim 34 in which said anchoring element incorporate a material or fabric to encourage cellular ingrowth.

37. The device of claim 36 in which said fabric comprises a Dacron, polyester, Gore-Tex, PTFE or other ingrowth encouraging material.

38. The device of claim 34 in which said anchored device comprises at least one complementary attachment site for the anchoring element.

39. The device of claim 38 in which said anchoring ring provides for firm attachment of said anchored device through use of an inflatable engaging member, shape-memory expansion, screwing, mechanical locking, magnetic attraction, chemical bonding, interference fitting or application of RF, EMF, ultrasonic or microwave energy.

40. The device of claim 38 in which said anchoring ring provides for firm attachment to said anchored device through the use of pins, staples, clips, stents, or sutures.

41. The device of claim 34 in which said anchoring element and said anchored device can each be collapsed to a size sufficient to allow for minimally invasive, catheter-based delivery.

42. The device of claim 34 in which radiographic contrast is incorporated into said anchored device to facilitate localization of anatomic landmarks (such as the coronary ostia) prior to placing said anchored device and also facilitate exact positioning/orientation of the implant.

43. The device of claim 34 in which said anchored device encourages native tissue ingrowth into the anchor with the embedding of elements in the anchor material that encourage cellular ingrowth.

44. The device of claim 34 in which the attachment of said anchoring device to said anchoring element is accompanied by another intervention, comprising native valve debridement for percutaneous aortic valve repair.

45. The device of claim 34 wherein said anchored device comprises one or more of the following devices: prosthetic aortic, tricuspid or mitral heart valves, abdominal aortic aneurysm stents, coronary stents, gastrointestinal stents, gastrointestinal devices anchored in an esophagus, stomach or duodenum, gastrointestinal devices anchored within a gastrointestinal lumen, urogynecologic devices anchored within a bladder, uterus, fallopian tubes or vagina, peritoneal devices anchored within a peritoneum, pulmonary devices anchored within a pulmonary tree, nasopharyngeal devices anchored within a nasopharynx, orthopedic devices anchored into bone and/or dermatologic devices anchored into skin.

46. A method of anchoring and removing an implanted device comprising:
placing an anchoring element attached to an anchored device wherein,
said anchoring element is reversibly engaged to said anchored device.

47. The method of claim 46 in which at least a portion of said anchoring element encourages native tissue ingrowth and/or cellular ingrowth.

48. The method of claim 46 in which said anchoring element comprises a ring, a tube, a socket, a port, a catheter, a patch, a clip, a suture, a staple, a socket, a region of magnetic attraction, chemical reaction, or energy delivery or any other fastener allowing for firm engagement of the complementary region of the anchored device.

49. The method of claim 46 in which said anchoring element contains support elements, comprising pins, staples, clips, stents, or sutures, to provide for firm attachment to the surrounding tissues at the site of implantation prior to said native tissue ingrowth and/or cellular ingrowth.

50. The method of claim 47 in which at least three days, at least a week, at least a month and/or at least three months is allowed to pass between the placement of said anchoring element and anchored device prior to detachment and removal of the anchored device.

51. The method of claim 50 in which the anchored device is detached from the anchoring ring and removed while the anchoring element is left in situ and not removed.

52. The method of claim 46 in which said anchoring ring provides for firm attachment to said anchored device through the use of screwing, mechanical locking, magnetic attraction, chemical bonding, interference fitting, suturing, stapling, or clipping.

53. The method of claim 46 wherein said anchored device comprises one or more of several devices including: prosthetic aortic, tricuspid or mitral heart valves, abdominal aortic aneurysm stents, coronary stents, gastrointestinal stents, electrical gastric stimulators, electrical intestinal stimulators, gastrointestinal devices anchored in an esophagus, stomach or duodenum, gastrointestinal devices anchored within a gastrointestinal lumen, urogynecologic devices anchored within a bladder, uterus, fallopian tubes or vagina, peritoneal devices anchored within a peritoneum, pulmonary devices anchored within a pulmonary tree, nasopharyngeal devices anchored within a nasopharynx, orthopedic devices anchored into bone and/or dermatologic devices anchored into skin.
